(19) United States
(12) Patent Application Publication
(10) Pub. No.: US 2019/0026422 A1
(43) Pub. Date: Jan. 24, 2019

(54) METHOD AND APPARATUS FOR DESIGNING PROTEINS

(71) Applicant: LONZA LTD, Visp (CH)

(72) Inventors: Ramon GOMEZ DE LA CUESTA, Thriplow, Hertfordshire (GB); Lars Arne Andreas ARNELL, Newmarket, Suffolk (GB); Jesús ZURDO, Cambridge, Cambridgeshire (GB); Laurent DUCRY, Sierre (CH); Laurence BONNAFOUX, Martigny (CH)

(21) Appl. No.: 15/550,237

(22) PCT Filed: Feb. 15, 2016
(86) PCT No.: PCT/EP2016/053167

§ 371 Date: Aug. 10, 2017

(30) Foreign Application Priority Data
Feb. 16, 2015 (GB) 1502590.1

(51) Int. Cl. G06F 19/16

(52) U.S. Cl. CPC G06F 19/16; A61K 47/6803

(57) ABSTRACT

Broadly speaking, the present invention provides a computer-implemented design method and a system to filter a large selection of mutated antibody sequences, identify those mutated antibody sequences which have particular desired properties, such that the identified sequences can be conjugated to a payload and tested in vitro. Thus, the design and system advantageously remove the need for physical testing of the entire initial selection of molecules, which is complex and costly. Only those which match the pre-defined design criteria may be subject to further experimental testing (in vitro testing) to confirm the results of the computer implemented design. Advantageously, by filtering unsafe molecules during the in silico design process, more time can be spent testing drugs which are predicted to be safe for humans.

### FIELD OF THE INVENTION

The invention relates to methods, system and apparatus for selecting antibody drug conjugates (ADCs).

### BACKGROUND TO THE INVENTION

Antibody-drug conjugates (ADCs) are a class of targeted therapies which combine the specificity of antibodies with the cytotoxicity of cytotoxic therapeutics. ADCs are primarily considered candidates for the treatment of various cancers. ADCs comprise an antibody linked to a therapeutic drug.

One problem with development of ADCs is the conjugation technology used; if drugs are conjugated non-selectively to cysteine or lysine residues in the antibody, then this can result in a heterogeneous mixture of ADCs. This approach leads to suboptimal safety and efficacy properties and makes optimization of the biological, physical and pharmacological properties of an ADC challenging. In particular, heterogeneity can be a problem with respect to the distribution of cytotoxins (that is, site of attachment), and the loading of cytotoxins (that is, number of drug molecules per antibody).

Heterogeneity presents safety concerns since high drug/antibody ratio (DAR) species can have poor binding to their target and increase risks of off-target toxicity. Low drug loading species are less active (DAR 1) or inactive (DAR 0). As the number of drugs per mAb decreases, the pharmacokinetic properties of the ADC improves (Hamblett, Clin. Cancer Res. 2004, 10, 7063-7070). Furthermore, heterogeneity of ADCs leads to challenges associated with consistent manufacturing and analytical testing.

Site-selective conjugation (SSC) would presumably improve ADCs' safety and efficacy, and thus, results in higher ADC quality. Junutula et al Nature Biotech 2008 report that same activity is achieved with half ADC dose in a SSC-ADC compared to control. Thus, ADC homogeneity will improve Therapeutic Index (TI is the ratio between maximum tolerated dose and effective dose. (TI=TD50/ED50)). Furthermore, higher DAR homogeneity would result in:
Simpler product characterization for regulatory filings, better defined product specifications;
Reduced off-target or bystander toxicity;
Potentially more controlled pharmacology;
Potentially reduce dosage requirements (per gram) to achieve similar pharmacological effect;
Reduced potential incidence of side-effects (immunogenicity, toxicity, etc.); and
Reduced costs due to higher conjugation yields and reduced dosage (required amount of ADC to be administered)

However, simply making antibodies with modified residues may result in unforeseen effects, such as changes to antibody aggregation propensity, solubility, or efficacy. The present applicants have recognised the need for improved methods and systems of selecting candidate antibody molecules which could be conjugated to a payload to produce ADCs with the desired properties.

### SUMMARY OF THE INVENTION

Broadly speaking, the present invention provides computer-implemented design methods and systems to filter a large selection of mutated antibody sequences, identify those mutated antibody sequences which have particular desired properties, such that the identified sequences can be conjugated to a payload and tested in vitro. Only those which match the pre-defined design criteria may be subject to further experimental testing (in vitro testing) to confirm the results of the computer implemented design. Such computer-implemented design may be termed in silico. Generally, it is desirable to design antibody sequences with specific mutations to which payloads (e.g. therapeutic drugs) can be conjugated. However, these mutated antibody sequences may not have the same or similar properties to the candidate (parental) antibody sequence. Antibodies with modified residues may have different antibody aggregation propensity, solubility, or efficacy, compared to the parental antibody sequence. Therefore, a method for selecting antibody sequences having the desired properties is required, prior to producing the ADCs.

A candidate antibody sequence (also referred to herein as the parent antibody sequence, or parental antibody sequence) is an antibody which is used as the basis for preparing modified antibodies.

A number of different criteria may be applied to a mutated antibody sequence to determine if it has the desired properties. For example: mutation site analysis may be used to determine if the mutated antibody sequences have similar physiochemical properties to the original candidate antibody sequence; aggregation propensity is a measure of how likely each mutated antibody sequence is to aggregate.

According to a first aspect of the present invention, there is provided a computer-implemented method of identifying one or more antibody sequences which are predicted to permit conjugation to a payload, each antibody sequence having a variable region which binds a target molecule and a constant region which comprises a mutated residue introducing a site specific conjugation site to permit conjugation of the antibody to a payload, the method comprising: inputting a candidate antibody sequence; identifying, using a mutation site analysis module, a plurality of mutated antibody sequences by: identifying at least one site in the candidate antibody sequence where a mutated residue is introducible; identifying at least one mutation which is introducible at each identified site; and introducing each identified mutation at each identified site to produce a plurality of mutated antibody sequences; calculating, using a solvent accessibility surface module, a value representative of solvent accessibility surface for each of the plurality of mutated antibody sequences; calculating, using an aggregation propensity module, a value representative of aggregation propensity for each of the plurality of mutated antibody sequences; comparing the calculated solvent accessibility value and the aggregation propensity value with the corresponding threshold values for solvent accessibility and aggregation propensity; filtering the plurality of mutated antibody sequences based on whether the threshold values for solvent accessibility and aggregation propensity are met; and outputting one or more mutated antibody sequences which are predicted to permit conjugation to a payload.

According to a second aspect of the present invention, there is provided a system for identifying one or more antibody sequences which are predicted to permit conjugation to a payload, each antibody sequence having a variable region which binds a target molecule and a constant region which comprises a mutated residue introducing a site specific conjugation site to permit conjugation of the antibody to a payload, the system comprising: an input device for inputting a candidate antibody sequence; a database comprising solvent accessibility surface and aggregation propensity data and corresponding threshold values thereof; at least one processor coupled to the input device and the database, wherein the processor is configured to: receive the input candidate antibody sequence; identify, using a mutation site analysis module, a plurality of mutated antibody sequences by: identifying at least one site in the candidate antibody sequence where a mutated residue is introducible; identifying at least one mutation which is introducible at each identified site; and introducing each identified mutation at each identified site to produce a plurality of mutated antibody sequences; calculate, using a solvent accessibility surface module, a value representative of solvent accessibility surface for each of the plurality of mutated antibody sequences; calculate, using an aggregation propensity module, a value representative of aggregation propensity for each of the plurality of mutated antibody sequences; compare the calculated solvent accessibility value and the aggregation propensity value with the corresponding threshold values for solvent accessibility and aggregation propensity; filter the plurality of mutated antibody sequences based on whether the threshold values for solvent accessibility and aggregation propensity are met; and output one or more mutated antibody sequences which are predicted to permit conjugation to a payload.

The following features apply equally to the methods and systems described above.

The system may be a computer and the method may be a computer-implemented method. The components of the system (e.g. processor, database and input) may be located in the same physical machine or the functionality may be spread across several interconnected machines. The processor is a hardware device such as a microprocessor which is configured to perform the steps of the method or as identified above. Again, the functionality may be provided in a single physical device or spread across several machines.

The term ‘mutation site analysis module’ as used herein refers to software running on any hardware device, where the software is configured to receive one or more physiochemical criteria as an input, and analyse one or more mutated antibody sequences, to determine if the or each mutated antibody sequence matches the or each physiochemical criterion. The mutation site analysis module (software) may be running on a hardware device that is dedicated to performing this function, or may be a hardware device which performs multiple functions, or a generic hardware device. The hardware device comprises a processor (which is defined above).

The term ‘aggregation propensity module’ as used herein refers to software running on any hardware device which is configured to receive one or more aggregation propensity criteria and analyse one or more mutated antibody sequences, to determine how likely each mutated antibody sequence is to aggregate, and if the or each aggregation propensity criterion is satisfied. The aggregation propensity module (software) may be a hardware device that is dedicated to performing this function, or may be a hardware device which performs multiple functions, or a generic hardware device. The module may comprise a processor (which is defined above).

In this way, the computer-implemented design and system may filter a large selection of candidate antibodies and mutated antibody sequences, and identify those mutated antibody sequences which have the desired properties, such that the identified sequences can be conjugated to a payload and tested in vitro. Thus, the design and system advantageously remove the need for physical testing of the entire initial selection of candidate molecules, which is complex and costly. Only those which match the pre-defined design criteria may be subject to further experimental testing (in vitro testing) to confirm the results of the computer implemented design. Such design may be termed in silico.

The term ‘antibody’ as used herein refers to any immunoglobulin, preferably a full-length immunoglobulin. Preferably, the term covers monoclonal antibodies, polyclonal antibodies, multispecific antibodies, such as bispecific antibodies, intracellular antibodies (or intrabodies) and antibody fragments thereof, so long as they exhibit the desired biological activity.

The term ‘antibody fragment’ or ‘fragment’ as used herein refers to a portion of a full-size (full-length) antibody which retains the specific binding properties of the antibody.

The term ‘derivative’ as used herein refers to a modified antibody or antibody fragment having one or more changes to the peptide sequence, and/or bearing one or more functional groups or one or more moieties bound thereto, which retains the specific binding properties of the antibody. A ‘derivative’ may include post-translationally modified antibodies.

The term ‘site specific conjugation sites’ as used herein refers to amino acid residues within an antibody which are specifically modified in order to permit conjugation of a payload.

The term ‘wild type’ as used herein refers to an unmodified, naturally occurring, peptide or nucleic acid sequence.

The term ‘parent antibody’ as used herein refers to an antibody which is used as the basis for preparing modified antibodies.

The term ‘non-natural amino acid’ as used herein refers to an amino acid that is not a proteinogenic amino acid, or a post-translationally modified variant thereof. In particular, the term refers to an amino acid that is not one of the 20 common amino acids

The term ‘antigen-binding fragment’ in the context of the present invention refers to a portion of a full length antibody where such antigen-binding fragments of antibodies retain the antigen-binding function of a corresponding full-length antibody.

By ‘antibody’ is meant any antigen-binding immunoglobulin molecule. The antibody is preferably a complete mammalian antibody (comprising two heavy chains and two light chains, each of which includes a constant region and a variable region), but other forms of antibody and derivative may be used. For example, Fabs, bi specific antibody fragments (tandem scFv-Fc, scFv-Fc knobs-into-holes, scFv-Fc-scFv, F(ab)₂, Fab-scFv, (Fab′scFv)₂, scDiabody-Fc, or scDiabody-C_{H}3), IgG-based bispecific antibodies (Hybrid hybridoma, Knobs-into-holes with common light chain, Two-in-one IgG, Dual V domain IgG, IgG-scFv, scFv-IgG, IgG-V, V—IgG), minibody, tribi-minibody, nanobodies, di-diabody and other non-Ig derived protein binding domains.

The one or more mutations preferably comprise mutation from a non-cysteine amino acid to a cysteine amino acid. Alternatively, the mutation may be to a lysine, glutamine, or a non-natural amino acid. Preferably the non-cysteine amino acid is selected from serine, valine, threonine, or alanine; more preferably serine or threonine. For example, the modification of antibodies with non-natural amino acids is described in WO2013/185115.

The antibody may be selected from Abciximab; Rituximab; Basiliximab; Daclizumab; Palivizumab; Infliximab; Trastuzumab; Alemtuzumab; Adalimumab; Efalizumab; Cetuximab; Ibritumomab; Omalizumab; Bevacizumab; Ranibizumab; Golimumab; Canakinumab; Ustekinumab; Tocilizumab; Ofatumumab; Belimumab; Ipilimumab; Brentuximab; Pertuzumab; Raxibacumab; Vedolizumab; Ramucirumab; Obinutuzumab; Siltuximab; Secukinumab; Dinutuximab.

The term ‘physicochemical properties’ as used herein for both the native and conjugated protein forms covers the molecular weight, molecular weight of the each constituent chain, the iso-electric point, charge and charge distribution along the sequence as well as across the structure and over the surface, hydrophobicity and hydrophobicity distribution along the sequence as well as across the structure and over the surface, the sequence secondary structure propensity for alpha-helices and beta-sheets, the native proteins covalent bond potential, hydrogen bond potential, unsatisfied buried hydrogen bonds, number of carbohydrate chains, carbohydrate chain content, structure and heterogeneity, solubility and stability at elevated temperatures or otherwise denaturing conditions. The term preferably also covers aggregation propensity, drug-to-antibody ratio, conjugation efficiency and protein-conjugate stability.

The term ‘biological properties’ as used herein covers the binding kinetics and reactivity of an immunoglobulin and/or an antibody drug conjugate. The term is also meant to refer to the native and conjugated protein forms half-life in serum, half-life in vivo and immunogenicity.

Generally speaking, it is desirable to design antibody sequences with specific cysteine mutations to which payloads (e.g. therapeutic drugs) can be conjugated. Alternatively, the mutation may be to a lysine, glutamine, or a non-natural amino acid. The antibodies incorporating the mutations are termed ‘mutated antibody sequences’ herein. The mutated antibody sequences may not have the same or similar properties to the candidate (parental) antibody sequence. As mentioned above, making antibodies with modified residues may result in unforeseen effects, such as changes to antibody aggregation propensity, solubility, or efficacy. Therefore, a method for selecting antibodies having the desired properties is required, prior to producing the ADCs.

In embodiments, the system is a computer system and comprises a mutation site analysis module, a solvent accessibility surface assessment module, and an aggregation propensity analysis module. One or more of these modules may be implemented as a separate machine, for example, coupled to the computer system over a network, or may comprise a separate or integrated programme running on the computer system.

Modelling the solvent accessibility surface (SAS), also referred to herein as the solvent accessible surface area, determines whether an antibody sequence (or mutated antibody sequence) is likely to conjugate with a therapeutic drug. Generally, SAS provides an indication of the approximate surface area of each candidate antibody that is accessible to a solvent. Thus, in embodiments, the SAS of a mutated antibody may preferably be no lower than the SAS of the candidate antibody, to ensure that the mutated antibody is as likely, or more likely, to conjugate with a therapeutic drug as the candidate antibody. In embodiments, the value representative of solvent accessibility surface for a specific mutated amino acid residue is a predicted percentage side chain solvent accessibility surface ratio which is given by:

$$100 \times \frac{S}{R}$$

where S is side chain solvent accessibility for a candidate amino acid residue, and R is side chain solvent accessibility of the mutated amino acid residue. Preferably, the threshold value for solvent accessibility surface is >15%. In embodiments, the threshold value for solvent accessibility surface is greater than 10%, 20%, 25%, 30%, 35%, 40%, 45%, 50%, 55%, 60%, 65%, 70%, 75%, 80%, 85%, 90%, 95%, or over 100%. In another embodiment the threshold value for solvent accessibility surface is between 25% and 76% or between 25% and 76%.

In embodiments, the outputting step comprises outputting mutated sequences having a predicted percentage side chain solvent accessibility surface of greater than or equal to the threshold value.

The aggregation propensity of each antibody sequence is modelled in silico to determine if they are likely to aggregate during expression or manufacturing and ultimately negatively affect the quality of the product.

In embodiments, the threshold value for aggregation propensity is calculated using a mean aggregation propensity for a set of reference antibody sequences, and wherein the outputting step comprises outputting mutated sequences which are predicted to have an aggregation propensity less than or equal to the threshold value. Aggregation prediction methods have been described by Pawar et al (Pawar, A. P., Dubay, K. F., Zurdo, J., Chiti, F., Vendruscolo, M., & Dobson, C. M. (2005), Prediction of “aggregation-prone” and “aggregation-susceptible” regions in proteins associated with neurodegenerative diseases, Journal of molecular biology, 350(2), 379-392), Bucciantini et al (Bucciantini, M., Giannoni, E., Chiti, F., Baroni, F., Formigli, L., Zurdo, J., . . . & Stefani, M. (2002), Inherent toxicity of aggregates implies a common mechanism for protein misfolding diseases, Nature, 416(6880), 507-511), and Tartaglia et al (Tartaglia, G. G., Cavalli, A. & Vendruscolo, M. (2007), Structure, 15, 139-143), as well as in patent applications such as U.S. Pat. No. 7,379,824, US2008/0262742, WO2004/0661168, WO2005/045442, WO2009/15518, and WO2009/068900A2.

As described in more detail below, the aggregation propensity is calculated based on a Z-score (also known as the standard score) comparison of the candidate (parental) antibody sequence and the mutated antibody sequence to the distribution of values for a reference set of the smallest functional domain of the antibody or protein where the mutation to cysteine is introduced. The set of reference antibody sequences contains those sequences having the same functional domain or domains for which the residue mutation screening/modelling is being performed. Specifically, the Z-score is the number of standard deviations a datum is above the mean (average) aggregation propensity value of a sample. A positive Z-score indicates a datum is above the mean, while a negative Z-score indicates a datum is below the mean. A mean and standard deviation is determined for the reference set. The Z-score is calculated by subtracting the reference mean from the target protein score and dividing by the standard deviation.

The result of the calculation is a zero (0) centred score where positive values indicate that the target is more aggregation prone (in this case) than the mean. Targets with a Z-score within (−1, 1) are within the standard deviation of the score within the reference set. Preferably, the aggregation propensity of each mutated molecule should not be significantly increased by the introduction of the intended engineered residue. Accordingly, in embodiments, those molecules which have a Z-score lying outside (−1, 1), i.e. lying outside one standard deviation from the mean aggregation propensity of the sample, are discarded.

The aggregation propensity analysis module may be configured to calculate a standard deviation value for the set of reference antibody molecules, and the outputting step comprises outputting mutated antibody sequences which are predicted to have an aggregation propensity value within one standard deviation from the mean aggregation propensity.

Optionally, the method further comprises filtering the antibody sequences based on immunogenicity propensity, which determines to what extent a given therapeutic protein may lead to an immune response in patients. Thus, in embodiments, the computer system may be configured to predict immunogenicity propensity of each mutated antibody; and compare the predicted immunogenicity propensity of each mutated antibody with immunogenicity propensity of the candidate antibody; wherein the outputting step of the method or system comprises outputting mutated antibody sequences having a substantially similar immunogenicity propensity to the candidate antibody, wherein substantially similar means that the immunogenicity propensity of a mutated antibody sequence is within 5%, 10%, 15%, or 20% of the value of the immunogenicity propensity of the candidate antibody sequence.

As mentioned earlier, the mutated antibody sequences may not necessarily exhibit the same or similar structural or physiochemical properties as the candidate (parental or unmutated) antibody sequence. Thus, in embodiments, the method further comprises predicting physiochemical properties of each mutated antibody sequence in order to select only those mutated antibody sequences which have similar structural or physiochemical properties as the candidate (parental) sequence. For example, mutations to cysteine should not create intra-chain hydrogen bonds leading to the alteration of the local environment and the properties of the protein. In embodiments therefore, the method comprises: comparing the predicted physiochemical properties to physiochemical properties of the candidate antibody sequence; and selecting mutated antibody sequences having predicted physiochemical properties which are substantially similar to the physiochemical properties of the candidate antibody.

In embodiments, filtering the mutated antibody sequences based on the solvent accessibility surface value is performed before filtering the mutated antibody sequences based on the aggregation propensity value.

In embodiments, filtering the mutated antibody sequences based on aggregation propensity value is performed before filtering the mutated antibody sequences based on solvent accessibility surface value.

According to a further aspect of the invention, there is provided a method of making an antibody suitable for conjugation comprising: selecting one of the mutated antibody sequences which is output from the above-described method; synthesising the sequence; and conjugating a payload to the selected, synthesised sequence. The method of synthesising the antibody sequences is described in more detail below.

In embodiments, the antibody is suitable for conjugation to a drug.

The term ‘synthesising the sequence’ used herein refers to methods of synthesising a gene sequence and generating the corresponding protein.

The payload may be a therapeutic drug molecule, for example. The drug payload may be a microtubule disrupting agent, or a DNA modifying agent. Examples of suitable drug payloads include dolastatin, vedotin, monomethyl auristatin F (MMAF), monomethyl auristatin E (MMAE), maytansinoids including DM1 and DM4, duocarmycin analogs, calicheamicin, pyrrolobenzodiazepines (PBD), duocarmycin, centanamycin, irinotecan, alpha-amanitin, and doxorubicin. Other drug payloads may be used, as may non-drug payloads (for example, the payload may be selected from the group consisting of a radionuclide, a chemotherapeutic agent, a cytotoxic agent, a microbial toxin, a plant toxin, melatonin, a membrane disrupting peptide, a polymer, a carbohydrate, a cytokine, a fluorescent label, a luminescent label, an enzyme-substrate label, an enzyme, a peptide, a peptidomimetic, a nucleotide, an siRNA, a microRNA, an RNA mimetic, and an aptamer).

The antibody, fragment, or derivative may be conjugated directly or indirectly to the drug payload; indirect conjugation may take place via a linker. The antibody may further comprise a linker. Suitable linkers include maleimide linkers, which permit conjugation to a payload via succinimide conjugation. The linker may be cleavable, or non-cleavable. Other suitable linkers are disclosed in international patent application WO2012/113847, which describes a branched linker which comprises a peptide chain and is derived from o-hydroxy p-amino benzylic alcohol, wherein the peptide chain is connected to the phenyl ring via the p-amino group, the drug is connected to the phenyl ring via the benzylic alcohol moiety, and the antibody is connected to the phenyl ring via the o-hydroxy group. The linker may be selected from 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio) pentanoate (SPP), N-succinimidyl 4-(N-maleimidomethyl) cyclohexane-1 carboxylate (SMCC), N-Succinimidyl, (4-iodo-acetyl) aminobenzoate (SIAB), SPDB, hydrazone, maleimidocaproyl and 6-maleimidocaproyl-valine-citrulline-p-aminobenyloxycarbonyl (MC-vc-PAB); or is a branched linker which comprises a peptide chain and is derived from o-hydroxy p-amino benzylic alcohol, wherein the peptide chain is connected to the phenyl ring via the p-amino group, the payload is connected to the phenyl ring via the benzylic alcohol moiety, and the antibody is connected to the phenyl ring via the o-hydroxy group.

According to a further aspect of the invention, there is provided a method of selecting one or more antibody drug conjugates, comprising: making at least one antibody drug conjugate using the above-mentioned method; performing one or more in vitro tests to determine biological and physiochemical properties of the antibody drug conjugates; comparing the determined biological and physiochemical properties with corresponding threshold values for the biological and physiochemical properties; and selecting the antibody drug conjugates based on whether the threshold values for the biological and physiochemical properties are met.

In embodiments of the method of selecting the physiochemical properties include a drug to antibody ratio (DAR) having a threshold value of between 85% and 110% of site specific conjugation sites per antibody, and wherein the selecting step comprises selecting antibody drug conjugates having a determined DAR value within the threshold value range. In a preferred embodiment, the DAR has a threshold value of at least 80%, at least 85%, at least 90%, at least 95%, or at least 100% of site specific conjugation sites per antibody.

In embodiments, the antibody comprises two site specific conjugation sites (i.e., one on either the heavy or light chain, and two such chains per antibody). Here, in embodiments the threshold value for DAR is between 1.7 to 2.2, and the selecting step comprises selecting antibody drug conjugates having a determined DAR value within the threshold value range. The DAR is preferably from 1.8-2.1, more preferably from 1.9-2.1, and most preferably around 2.0.

Additionally or alternatively, in embodiments the antibody comprises four site specific conjugation sites (i.e., one on the heavy chain and one on the light chain, or two on the heavy chain and none on the light chain, or none on the heavy chain and two on the light chain, and two such chains per antibody). For such an antibody, the DAR is also preferably between 85% and 110%, such that the threshold value of DAR is between 3.4 and 4.4. In embodiments, the value of DAR is preferably between 3.6 and 4.2, more preferably between 3.8 and 4.2, and most preferably 4.

In embodiments, the physiochemical properties include a value for conjugation efficiency, wherein the threshold value for conjugation efficiency is at least 80%, and wherein the selecting step comprises selecting antibody drug conjugates having a determined conjugation efficiency greater than or equal to the threshold value. In embodiments, conjugation efficiency is between 85% and 110% of the site specific conjugation. Thus, in embodiments, the threshold value for conjugation efficiency is at least 80%, 85%, 90%, 95%, 100%, or 105%.

In embodiments, the physiochemical properties include a value for protein-conjugate stability, wherein the threshold value for stability is at least 60% for a pre-defined period of time, and wherein the selecting step comprises selecting antibody drug conjugates having a determined stability greater than or equal to the threshold value. In particular, the term “protein-conjugate stability” used herein means the level of deconjugation. In embodiments, when stability of an antibody drug conjugate is analysed in vitro, the percentage average loss of conjugated molecule over a period of 8 days is preferably <39%. In embodiments, the threshold value for stability is at least 50%, 55%, 60%, 65%, 70%, 75%, 80%, 85%, 90%, or over 95%. In embodiments, the percentage average loss of the conjugated molecule over a specific period of time is preferably less than 5%, 10%, 15%, 20%, 25%, 30%, 35%, 40%, 45% or 50%.

In embodiments of the system the solvent accessibility surface module is configured to: receive the identified plurality of mutated antibody sequences from the processor; request the solvent accessibility surface threshold value from the database; and calculate the value representative of solvent accessibility surface for each mutated antibody sequence; wherein the value representative of solvent accessibility surface for is a predicted percentage side chain solvent accessibility surface ratio for each mutated antibody sequence given by:

$$100 \times \frac{S}{R}$$

where S is side chain solvent accessibility for a candidate amino acid residue, and R is side chain solvent accessibility of a specific mutated antibody sequence.

In embodiments of the system the threshold value for solvent accessibility surface is 15%, and wherein the solvent accessibility surface module is configured to: filter the received identified plurality of mutated antibody sequences; and output those mutated antibody sequences having a solvent accessibility surface value greater than the threshold value. In embodiments, the threshold value for solvent accessibility surface is greater than 10%, 20%, 25%, 30%, 35%, 40%, 45%, 50%, 55%, 60%, 65%, 70%, 75%, 80%, 85%, 90%, 95%, or over 100%.

In embodiments of the system the aggregation propensity analysis module is configured to: receive the identified plurality of mutated antibody sequences from the processor; request a set of reference antibody sequences from the database; calculate the aggregation propensity threshold value by calculating the mean aggregation propensity for the set of reference antibody sequences; calculate a value representative of aggregation propensity for each received mutated antibody sequence; filter the received identified plurality of mutated antibody sequences; and output those mutated antibody sequences having an aggregation propensity value less than or equal to the threshold value.

In embodiments of the system the aggregation propensity analysis module is further configured to: calculate a standard deviation value for the set of reference antibody sequences; and filter the received identified plurality of mutated antibody sequences; and output those mutated antibody sequences having an aggregation propensity value within one standard deviation from the mean aggregation propensity.

In embodiments of the system, the at least one processor is configured to filter the mutated antibodies based on solvent accessibility surface criteria before filtering the mutated antibodies based on aggregation propensity criteria.

In embodiments of the system, the at least one processor is configured to filter the mutated antibodies based on aggregation propensity criteria before filtering the mutated antibodies based on solvent accessibility surface criteria.

According to a further aspect of the invention, there is provided a method of making a pharmaceutical composition wherein the composition comprises at least one antibody or antibody fragment having a variable region which binds a target molecule and a constant region, wherein the constant region comprises one or more mutations introducing a site specific conjugation site to permit conjugation of the antibody to a payload, wherein the at least one antibody or antibody fragment is identified by the method of described herein and the composition is formulated with a pharmaceutically acceptable carrier, adjuvant and/or excipient. Pharmaceutical compositions of the present invention can also be administered as part of a combination therapy, meaning the composition is administered with at least one other therapeutic agent, for example, an anti-cancer drug.

A pharmaceutically acceptable carrier can include solvents, dispersion media, coatings, antibacterial and antifungal agents, isotonic and absorption delaying agents. Preferably the carrier is suitable for intravenous, intramuscular, subcutaneous, parenteral, spinal or epidermal administration.

The pharmaceutical compositions of the present invention may also include one or more pharmaceutically acceptable salts, a pharmaceutically acceptable anti-oxidant, excipients and/or adjuvants such as wetting agents, emulsifying agents and dispersing agents.

According to a further aspect of the invention, there is provided a carrier carrying code which when implemented on a processor causes said processor to carry out the steps of the method described herein.

The or each processor may be implemented in any known suitable hardware such as a microprocessor, a Digital Signal Processing (DSP) chip, an Application Specific Integrated Circuit (ASIC), Field Programmable Gate Arrays (FPGAs), etc. The or each processor may include one or more processing cores with each core configured to perform independently. The or each processor may have connectivity to a bus to execute instructions and process information stored in, for example, a memory.

The invention further provides processor control code to implement the above-described systems and methods, for example on a general purpose computer system or on a digital signal processor (DSP). The invention also provides a carrier carrying processor control code to, when running, implement any of the above methods, in particular on a non-transitory data carrier—such as a disk, microprocessor, CD- or DVD-ROM, programmed memory such as read-only memory (Firmware), or on a data carrier such as an optical or electrical signal carrier. The code may be provided on a carrier such as a disk, a microprocessor, CD- or DVD-ROM, programmed memory such as non-volatile memory (e.g. Flash) or read-only memory (Firmware). Code (and/or data) to implement embodiments of the invention may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog™ or VHDL (Very high speed integrated circuit Hardware Description Language). As the skilled person will appreciate such code and/or data may be distributed between a plurality of coupled components in communication with one another. The invention may comprise a controller which includes a microprocessor, working memory and program memory coupled to one or more of the components of the system.

According to a related aspect of the invention, there is provided a method for selecting a residue of an antibody sequence (or fragment or derivative) for mutation to any one of cysteine, lysine, glutamine, or a non-natural amino acid, comprising:
calculating a value representative of solvent accessibility surface (SAS) for the antibody sequence;
calculating the aggregation propensity of the antibody sequence; and
selecting an amino acid residue of the antibody sequence for mutation if the SAS is greater than a threshold value of SAS and the aggregation propensity is less than or equal to a threshold value of aggregation propensity.

### BRIEF DESCRIPTION OF THE DRAWINGS

The invention is diagrammatically illustrated, by way of example, in the accompanying drawings, in which:

FIG. 1 is a schematic of a site-selective conjugation process to produce antibody drug conjugates, according to an embodiment of the invention;

FIG. 2a shows a flow chart of the process to select antibody sequences to facilitate site-selective conjugation, in an embodiment of the invention;

FIG. 2b shows a more detailed flow chart of the process to select antibody sequences, in an embodiment of the invention;

FIG. 3 shows a block diagram of a computer system for implementing an embodiment of the invention;

FIG. 4 shows levels of expression of candidate antibody in 25 ml CHOK1 SV cultures;

FIG. 5 shows the percentage monomer for conjugated and nonconjugated molecules, and the results from SEC-HPLC analysis of aggregation and fragmentation propensity for conjugated molecules;

FIGS. 6a and 6b show respectively, the extent of biotin-maleimide conjugation to light and heavy chains of the mutated antibody drug sequences; open bars represent unconjugated product, striped bars represent products with a single conjugated payload, and solid bars represent products with two conjugated payloads

FIG. 7 shows the calculated drug to antibody ratio of the mutated antibody drug sequences; and

FIGS. 8a and 8b respectively show the percentage biotin decrease over 8 days and the average percentage biotin decrease.

### DETAILED DESCRIPTION OF THE DRAWINGS

Broadly speaking, the present invention provides a computer-implemented design method and a system to filter a large selection of mutated antibody sequences, identify those mutated antibody sequences which have particular desired properties, such that the identified sequences can be conjugated to a payload and tested in vitro. Thus, the design and system advantageously remove the need for physical testing of the entire initial selection of molecules, which is complex and costly. Only those which match the pre-defined design criteria may be subject to further experimental testing (in vitro testing) to confirm the results of the computer implemented design. Such computer-implemented design may be termed in silico. Advantageously, the in silico design process enables antibody drug conjugates to be selected for further testing only if they match certain criteria which are required in a drug that is to be used in patients. By filtering unsafe molecules during the in silico design process, more time can be spent testing drugs which are predicted to be safe for humans.

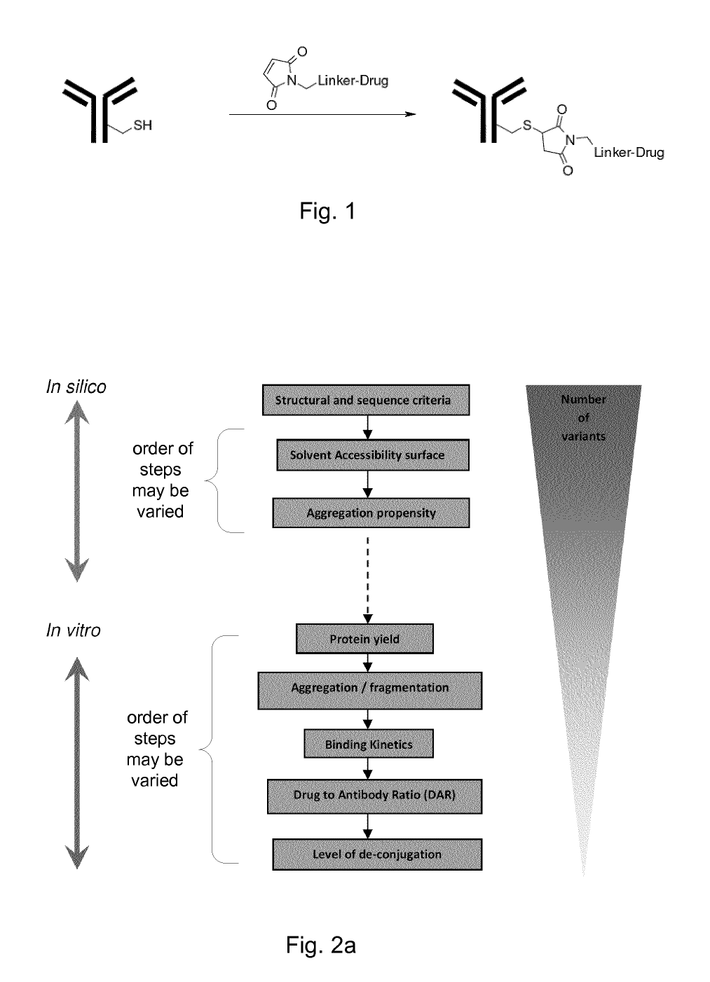

FIG. 1 shows a schematic of a site-selective conjugation process to produce antibody drug conjugates (ADCs). In embodiments, it is desirable to design antibody sequences with specific cysteine mutations to which payloads (e.g. therapeutic drugs) can be conjugated. However, these mutated antibody sequences may not have the same or similar properties to the candidate (parental) antibody sequence. As mentioned above, making antibodies with modified residues may result in unforeseen effects, such as changes to antibody aggregation propensity, solubility, or efficacy. Therefore, a method for selecting antibody sequences having the desired properties is required, prior to producing the ADCs.

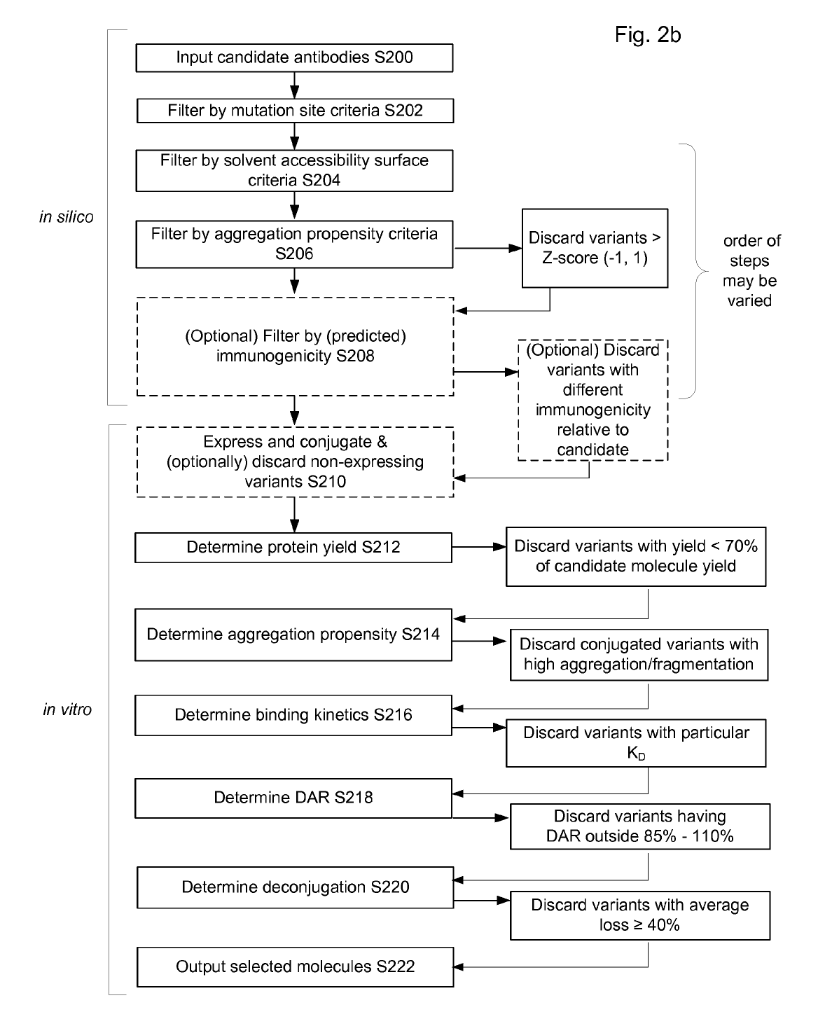

FIG. 2a shows a general flow chart of the process to select antibody variants (i.e. mutated antibody drug sequences) to facilitate site-selective conjugation, in an embodiment of the invention. The selected antibodies or antibody fragments (e.g. the fragment antigen-binding (Fab) regions on an antibody which binds to antigens) contain a number of amino acid substitutions to facilitate site-specific conjugation to a bioactive payload of choice. In particular, the selected molecules are chosen following a series of in silico and in vitro tests performed on a set of candidate antibody sequences. Preferably, these tests are a series of sequentially-performed tests, such that only those candidate antibody variants that satisfy the criterion within each step are progressed to the next step in the selection process. Consequently, the antibody sequences which remain at the end of the selection process generally share a number of specific properties (i.e. conform to a set of design criteria).

Generally speaking, the design process incorporates a number of in silico and in vitro screening steps. Thus, the antibody sequences of the present invention share a number of properties, as described in more detail below. As shown in FIG. 2a, the first step in the selection process, is to identify mutated antibody sequences (i.e. antibody sequences having a mutated residue). This may therefore be considered a preparatory step performed prior to the filtering/selection process. A candidate antibody is assessed to identify regions which could be mutated to introduce a cysteine mutation. The mutated residues should have similar physiochemical properties to the original candidate (parental) antibody molecule. Thus, the mutation site criteria (i.e. structural and sequence criteria) are used to identify suitable mutated antibody sequences for the selection process.

FIG. 2a shows how the in silico filtering steps generally comprise screening the identified mutated molecules on the basis of solvent accessibility surface criteria and aggregation propensity. Modelling the solvent accessibility surface (SAS, also known as the solvent accessible surface area) determines the approximate surface area of each candidate antibody that is accessible to a solvent. This is important because it indicates whether a molecule is likely to conjugate with a therapeutic drug. The aggregation propensity of each candidate is modelled to determine if they are likely to aggregate in their folded state and ultimately form organised structures.

The order of the screening steps can be varied and is not limited to the specific order shown in FIG. 2a. For example, it may be preferable to screen based on aggregation propensity first, if this is considered a particularly important property of the selected molecules, which could therefore be used as a first filter of the candidate antibody sequences. Changing the order of the in silico screening steps may or may not affect the subset of the candidate antibody sequences which proceed to the in vitro part of the process.

The in vitro part of the process tests the subset of candidate antibody sequences which have successfully passed the in silico screening. Generally speaking, the in vitro tests are used to select antibody molecules which have particular properties, such as high protein yield, a particular drug-to-antibody ratio (DAR) and high stability. These tests are described in more detail below with respect to FIG. 2b. At the end of the process, only those molecules remain which satisfy all of the in silico and in vitro design criteria. Further analysis may be performed on the selected molecules.

Turning now to FIG. 2b, this shows a more detailed flow chart of the process to select antibody sequences, in an embodiment of the invention.

### Structural and Sequence Assessment (in Silico)

The process begins by selecting candidate antibody sequences (S200) and performing a preparatory step to identify a subset of the candidate antibody sequences which could be mutated to introduce a cysteine mutation and which, when mutated, have similar physiochemical properties to the original candidate (parental) antibody molecule (S202).

Ser, Thr, Val, and Ala residues in CH1, CH3, and CL were explored. This gave a number of candidates:

Light chain: SER171, VAL191, SER208, SER182, THR180, THR206, ALA184, SER203, SER202 Heavy chain: VAL170, VAL176, THR190, SER445, SER443, SER139, SER160, SER447, THR200, SER168.

(The residues are identified using a sequential numbering system. By “positional numbering”, “sequential numbering” and similar terms is meant the numbering of the amino acid sequence of the peptide in which the first residue at the N terminus is designated residue number 1, and subsequent residues are sequentially numbered residue 2, 3, 4 . . . etc. This is contrasted with Kabat or EU numbering systems for antibodies).

Preferably, the residues to be mutated to cysteine must have similar physicochemical properties or be a small non-hydrophobic non-charged residue (e.g. serine, valine, threonine, alanine). The residues amenable to be mutated to cysteine must be in constant regions of either the light chain (C_{K}, C_{λ},) or the heavy chain (C_{H}1, C_{H}2 or C_{H}3) of an antibody or a fragment thereof (Fab).

Mutations to cysteine must not be placed in the interfaces between chains or domains of the antibody (or scaffold). In cases where a modified antibody or scaffold is used, the introduced cysteine is ideally at a distance of >5 Å from any target-binding interface or domain, to minimise the risk of interfering with the biological activity of the molecule. Introduced cysteine should be at a distance >5 Å from any antibody native cysteine and should not interfere with the Fc glycosylation site (i.e. should be placed at a distance >5 Å from residue Asn295 where glycosylation occurs).

Mutations to cysteine should not create intra-chain hydrogen bonds leading to the alteration of the local environment and the properties of the protein. Introduced cysteine should not increase the chemical degradation risk/should not introduce undesired post translational modifications.

Preferably, the structural stability of the candidate (parental) antibody is preserved in the mutated antibody drug sequences. The introduction of a cysteine residue in the desired position should not destabilise significantly the molecule. Thus, the introduced cysteine should not: disrupt or lead to the formation of intra-chain hydrogen bonds; lead to the formation of salt bridges or covalent bonds within the molecule; or interfere with the quaternary assembly of the molecule (i.e. inter-domain interfaces of an antibody).

For antibodies, the introduced cysteine residue should be positioned away from inter-domain and inter-chain regions of the antibody. Conjugation to engineered cysteine residues should not destabilise the molecule significantly, or increase aggregation or formation of ‘half-antibodies’.

Similarly, the introduction of a cysteine residue should have a low chemical degradation risk, and/or should not give rise to undesired post-translational modifications. Chemical degradation includes: oxidation, deamidation, proteolytic cleavage, etc. Posttranslational modifications include: phosphorylation, unintended glycosylation, ubiquitination, nitrosylation, methylation, acetylation, etc.

### Solvent Accessibility Surface Assessment (in Silico)

The in silico filtering comprises screening the identified mutated molecules on the basis of solvent accessibility surface criteria and aggregation propensity, and optionally on the basis of predicted immunogenicity.

As mentioned above, modelling the solvent accessibility surface (SAS) determines whether a mutated molecule is likely to conjugate with a therapeutic drug. Solvent accessibility surface modelling (step S204) was performed using Discovery Studio software (Accelrys Software Inc., Discovery Studio Modeling Environment, Release 4.0, San Diego: Accelrys Software Inc., 2013.) to calculate the side chain solvent accessibility surface of the chosen residues. Preferably, the SAS is >15% (more preferably >17%) for a molecule which is amenable to conjugation with a payload such as a therapeutic drug molecule. However, in embodiments in which a different parent (candidate) molecule is the starting point for the in silico screening, and/or a different residue is introduced into the parent molecule to generate mutated molecules, the SAS may be greater than 20%, 25%, 30%, 35%, 40%, 45%, 50%, 55%, 60%, 65%, 70%, 75%, 80%, 85%, 90%, 95%, 100%, 110%, 120%, 130%, 140%, etc. The percentage side chain solvent accessibility surface for a specific amino acid residue is calculated as 100 times the side chain solvent accessibility (S), divided by the side chain solvent accessibility of the specific amino acid residue (R, calculated using the extended Ala-X-Ala tripeptide, where X is the residue of interest):

$$100 \times \frac{S}{R}$$

In this example where the residue of interest is cysteine and the parent molecules are selected from the above-mentioned candidate antibody sequences, side chains with solvent accessibility ratios ≤17% are considered buried and are therefore discarded. Those side chains with SAS >17% proceed to the next step of the selection process.

However, if a different residue is selected, and/or different candidate antibody sequences are chosen, the SAS threshold value may be, for example, greater than 10%, greater than 20%, greater than 25%, etc. The results of the in silico SAS modelling on the above-mentioned variants are shown below. The variant (mutated antibody sequences) having a SAS value below the threshold value is shown underlined in the table. In the table, the SAS value of the VAL170 (heavy chain) variant is judged to be above the SAS threshold value, particularly since there is a big gap in terms of exposure between SER171 and VAL170. VAL170 is closer to VAL176 and THR190 so VAL170 is taken to be a variant with a SAS value above the threshold.

| Variant (LC) | % SAS | Variant (HC) | % SAS |
|---|---|---|---|
| SER171 | 5.45 | VAL170 | 17.74 |
| VAL191 | 48.069 | VAL176 | 25.179 |
| SER208 | 48.585 | THR190 | 27.548 |
| SER182 | 67.216 | SER445 | 33.961 |
| THR180 | 67.518 | SER443 | 46.173 |
| THR206 | 75.247 | SER139 | 53.401 |
| ALA184 | 116.94 | SER160 | 61.951 |
| SER203 | 122.689 | SER447 | 72.323 |
| SER202 | 136.452 | THR200 | 91.354 |
| | | SER168 | 133.127 |

Preferably, variant molecules have good reactivity—this might be parameterized with an upper and lower limit of the solvent accessibility surface, and validated in vitro.

Preferably, there is no di-sulphide scrambling. The introduction of a cysteine residue in the desired position should not lead to the formation of non-native disulfide bonds from disulfide scrambling

### Aggregation Propensity Assessment (in Silico)

The aggregation propensity of each candidate is modelled in silico to determine if they are likely to aggregate (S206). Preferably, the aggregation propensity of each mutated molecule should not be significantly increased by the introduction of the intended engineered cysteine residue. An increased risk of aggregation caused by the covalent bond potential of the introduced cysteine is expected. However, the in silico predicted intrinsic aggregation propensity should not increase significantly relative to the parental molecule.

Aggregation prediction methods have been described by Pawar et al (Pawar, A. P., Dubay, K. F., Zurdo, J., Chiti, F., Vendruscolo, M., & Dobson, C. M. (2005), Prediction of “aggregation-prone” and “aggregation-susceptible” regions in proteins associated with neurodegenerative diseases, Journal of molecular biology, 350(2), 379-392), Bucciantini et al (Bucciantini, M., Giannoni, E., Chiti, F., Baroni, F., Formigli, L., Zurdo, J., . . . & Stefani, M. (2002), Inherent toxicity of aggregates implies a common mechanism for protein misfolding diseases, Nature, 416(6880), 507-511), and Tartaglia et al (Tartaglia, G. G., Cavalli, A. & Vendruscolo, M. (2007), Structure, 15, 139-143), as well as in patent applications such as U.S. Pat. No. 7,379,824, US2008/0262742 and WO2009/068900A2.

The propensity to aggregate of the variants generated in the absence of any conformational restriction should preferably be neutral or reduced. The aggregation propensity is calculated based on a Z-score (also known as the standard score) comparison of the candidate (parental) molecule and the variants to the distribution of values for a reference set of the smallest functional domain of the antibody or protein where the mutation to cysteine is introduced. The set of reference antibody sequences contains those sequences having the same functional domain or domains for which the substitution screening/modelling is being performed.

The Z-score is the number of standard deviations a datum is above the mean value of a sample—a positive Z-score indicates a datum is above the mean, while a negative Z-score indicates a datum is below the mean. A mean and standard deviation is determined for the reference set. The Z-score is calculated by subtracting the reference mean from the target protein score and dividing by the standard deviation.

Consider the physicochemical aggregation propensity function to be P_{agg} and Z_{agg} to be the overall aggregation propensity. P_{agg} and Z_{agg} are described in the above-referenced paper by Pawar et al, and the above-referenced patent publications. However, P_{agg} can also be any general protein aggregation prediction algorithm. The reference mean is Pₘₑₐₙ, which may be calculated either:
as the average aggregation propensity of a random set sequences of the same length; or
using a set of similar proteins or domains with known aggregation behaviour.

Both the target protein score (P_{agg}) and each reference mean (Pₘₑₐₙ) is calculated in the same way.

The result is a zero (0) centred score where positive values indicated that the target is more aggregation prone (in this case) than the mean. Targets with a Z-score within (−1, 1) are within the standard deviation of the score within the reference set. The AggreSolve® in silico platform (Lonza, Basel, Switzerland) comprises a collection of algorithms which, based on sequence and structural parameters, can calculate predictors that reflect the aggregation propensity of a given polypeptide. (The platform uses aggregation prediction methods such as those described in the above-mentioned publications). Such predictors reflect global and local (residue-specific) aggregation propensities as well as local flexibility and stability. Accordingly, those molecules which have a Z-score lying outside (−1, 1) are discarded.

In embodiments, a restriction on the increase in the local (smallest functional domain) aggregation propensity is better than using a protein level score. It is, in general, more likely that the local aggregation profile is used to determine the aggregation propensity, rather than the full protein level. The local aggregation profile is also more likely to change upon a substitution, whereas the single point substitution in a large protein might only result in a negligible variation in the protein level score.

The results of the in silico aggregation propensity modelling are shown in the table below. The AggreSolve® Z-score has been calculated for the full length Trastuzumab heavy and light chain, as well as for the CH1, CH3, and CL domains in which the mutations/substitutions are located (i.e. the minimal functional domains). The boundaries for the CH1, CH3 and CL domains are as per the International ImMunoGeneTics information system, or IMGT®, definition in M. P. Lefranc, C. Pommie, Q. Kaas, E. Duprat, N. Bosc, D. Guiraudou, C. Jean, M. Ruiz, I. Da Piedade, M. Rouard, E. Foulquier, V. Thouvenin, and G. Lefranc, IMGT unique numbering for immunoglobulin and T cell receptor constant domains and Ig superfamily C-like domains (Developmental and comparative immunology 29 (3), 2005).

| | | Difference |
|---|---|---|
| Antibody Name | Z-score | (Variant − WT) |
| Trastuzumab Heavy Chain (H) | 0.36 | |
| H:S160C | 0.02 | −0.34 |
| H:T190C | 0.36 | 0.00 |
| H:S443C | 0.19 | −0.17 |
| H:S447C | 0.19 | −0.17 |
| Trastuzumab Light Chain (L) | 3.09 | |
| L:T180C | 2.85 | −0.24 |
| L:T206C | 3.10 | 0.01 |
| Trastuzumab Heavy Chain constant domain 1 | 0.77 | |
| (CH1) | | |
| CH1:S160C | 0.17 | −0.60 |
| CH1:T190C | 0.80 | 0.04 |
| Trastuzumab Heavy Chain constant domain 3 | −0.07 | |
| (CH3) | | |
| CH3:S443C | −0.33 | −0.26 |
| CH3:S447C | −0.36 | −0.29 |
| Trastuzumab Light Chain constant domain | 1.95 | |
| (CL) | | |
| CL:T180C | 1.66 | −0.29 |
| CL:T206C | 1.98 | 0.03 |

### Optional: Immunogenicity Assessment (in Silico)

Optionally, the molecules which pass the SAS and aggregation propensity tests may be further filtered by predicted immunogenicity (S208). The immunogenicity tests assess to what extent a given therapeutic protein may lead to an immune response in patients. When modelling immunogenicity propensity of antibody molecules, the most potentially immunogenic portion of the molecule resides in the hyper variable regions. The constant regions of the antibody molecules are recognised as “self” by the immune system and therefore, do not trigger immunogenic responses. Any mutation introduced in the constant regions will alter the region such that it is not recognised as “self”, which may therefore increase the immunogenicity of the molecule. Preferably, the immunogenicity of the variants is similar to that of the original (parental) molecule. However, this filtering step is an optional step because it is possible that the modelled immunogenicity of the variants pre-conjugation is dissimilar to the immunogenicity of the antibody variant in its active form (i.e. the variant conjugated to a payload).

As previously mentioned, the order of the in silico screening steps can be varied and not limited to the specific order shown in FIG. 2b. Changing the order of the in silico screening steps may or may not affect the subset of the candidate antibody sequences which proceed to the in vitro part of the process.

### Expression Assessment (In Vitro)

The in vitro part of the process tests the subset of mutated antibody sequences which have successfully passed the in silico screening tests. Generally speaking, the in vitro tests are used to select antibody molecules which have particular properties, such as high protein yield, a particular drug-to-antibody ratio (DAR) and high stability. Although a specific order of in vitro steps is shown in FIG. 2b, it will be understood that the order of steps used may be varied and is not limited to the specific order shown in the Figures or described herein. Optionally, the first in vitro test (S210) may be to express the antibody variants on a small scale to quickly check first, if the variants do express, and second, if the aggregation propensity and SAS data is similar to the modelled, expected values. Those variants which do not express and/or substantially match the expected aggregation propensity and SAS values are discarded. (However, expression of the variants is not essential and could be avoided if other resources/data are available.)

Antibodies were expressed for in vitro tests by gene synthesis, DNA amplification, and expression of the antibodies in CHO K1 SV transient transfection cultures. A construct may be used in an expression system in order to express an antibody (also known as an immunoglobulin).

Systems for cloning and expression of antibodies in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host for small immunoglobulin molecules is E. coli. The expression of immunoglobulins, such as antibodies and antibody fragments, in prokaryotic cells such as E. coli is well established in the art. Expression in eukaryotic cells in culture is also available to those skilled in the art as an option for production of a immunoglobulin. Immunoglobulins, such as antibodies and antibody fragments, may also be expressed in cell-free systems.

Suitable vectors for the expression of immunoglobulins can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. Nucleic acid encoding a variant immunoglobulin or a CH1, VH and/or VL domain thereof may be contained in a host cell.

Variant antibodies were generated as described for example in WO2011021009A1. In detail: DNA encoding the antibody variants as described herein were chemically synthesized and cloned into a suitable mammalian expression vector. For transient expression experiments heavy and light chain were cloned into separate expression vectors. For generation of cell lines stably expressing a variant antibody heavy and light chains were cloned into one single expression vector. Each expression vector comprises a DNA encoding a signal sequence upstream of the heavy chain and the light chain coding regions to enable secretion of the heavy and light chain from the mammalian cells.

For transient expression, CHOK1 SV cells were transfected using for example Lipofectamine with the expression vectors encoding the variants as described herein. For example in case of variants comprising at least one mutation in the light chain, an expression vector comprising said mutation(s) was co-transfected with a vector encoding the unmodified heavy chain; in case of variants comprising at least one mutation in the heavy chain, an expression vector comprising said mutation(s) was co-transfected with a vector encoding the unmodified light chain. 72 h post-transfection, supernatants were harvested form the transfected cells, centrifuged and stored at 4° C. prior to purification.

For Large scale production CHOK1 SV cells are transfected as described above with a single vector comprising modified or unmodified light and heavy chain. Either pools of stably transfected cell are used for further experiments or a clonal selection is performed. Supernatants of such stable transfected cells expressing a variant of the present invention was harvested and stored at 4° C. prior to purification.

Cell culture supernatants were Protein A purified using HiTrap columns (GE) and stored at 4° C. prior to concentration and buffer exchange. Samples were concentrated by centrifugation at 2000 g 15-20 min. Material was buffer exchanged 4-5 times using formulation buffer (50 mM Phosphate, 100 mM NaCl, pH7.4). Once buffer exchanged, samples were diluted in formulation buffer to an appropriate working concentration.

In embodiments, the expression method may comprise introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

The introduction may be followed by causing or allowing expression from the nucleic acid, e.g. by culturing host cells under conditions for expression of the gene. Nucleic acid encoding the variant immunoglobulin or CH1, VH and/or VL domain thereof may be integrated into the genome (e.g. chromosome) of the host cell. Integration may be promoted by inclusion of sequences which promote recombination with the genome, in accordance with standard techniques. Following production by expression, a variant immunoglobulin or CH1, VH and/or VL domain thereof may be isolated and/or purified using any suitable technique, then used as appropriate. For example, a method of production may further comprise formulating the product into a composition including at least one additional component, such as a pharmaceutically acceptable excipient.

Conjugation was carried out with biotin-maleimide conjugation to free thiol groups by standard techniques, such as that described by Junutula J R et al, Nature Biotechnology 2008, 8, 925-932 and Jeffrey S C et al, Bioconjugate Chem. 2013, 24, 1256-1263.

In an example of toxin conjugation, engineered antibodies are reduced with a tris(2-carboxyethyl)phosphine (12.5 eq.) for 2 h at 35° C. and pH 7.7. The mixture is buffer exchanged into 50 mM Tris, 5 mM EDTA, pH 7.7. Dehydroascorbic acid (15 eq.) is added and the oxidation reaction allowed to proceed for 3 h at 24° C. N,N-dimethylacetamide is added to reach a concentration of 5%. Maleimidocaproyl-valine-citrulline-p-aminobenzyloxycarbonyl-monomethylauristatin E (5 eq.) is added and the conjugation reaction allowed to proceed for 1 h at 22° C. The reaction is quenched by addition of N-acetyl-cysteine (5 eq.). Following 0.5 h incubation at 22° C., the mixture is buffered exchanged into 1×PBS.

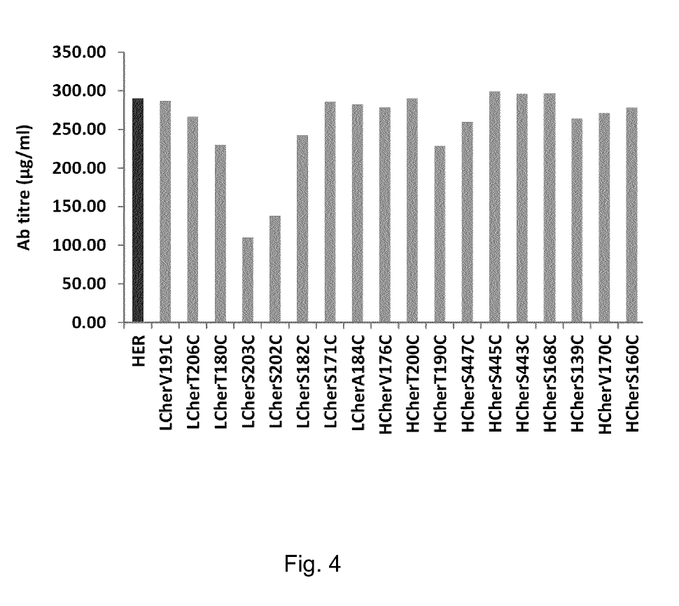

FIG. 4 shows levels of expression of each candidate antibody in 25 ml CHOK1 SV cultures. All variants show similar levels of expression, with the exception of the light chain variants S202C and S203C.

Preferably, the in vitro tests performed on the variants are used to determine protein yield (in vitro), aggregation/fragmentation, and binding kinetics.

### Protein Yield Assessment (In Vitro)

The introduction of a cysteine residue in the desired position should not impact negatively the product titre in supernatant or recovery after purification. The protein yield is estimated by product titre in supernatant and after protein A purification (e.g. through ELISA, with absorbance at 280 nm, or via HPLC protein A quantification). The protein yield is ideally >70% of the yield of the parental molecule and those variants with a low yield are discarded (step S212).

### Aggregation Propensity/Fragmentation Assessment (In Vitro)

The introduction of a cysteine residue in the desired position should not increase significantly the levels of aggregation.

Aggregation/fragmentation propensity was analysed via PAGE analysis (polyacrylamide gel electrophoresis), preferably SDS (sodium dodecyl sulphate) PAGE. Each variant antibody was treated with beta mercaptoethanol, or given no treatment, and size fractionated on a gel.

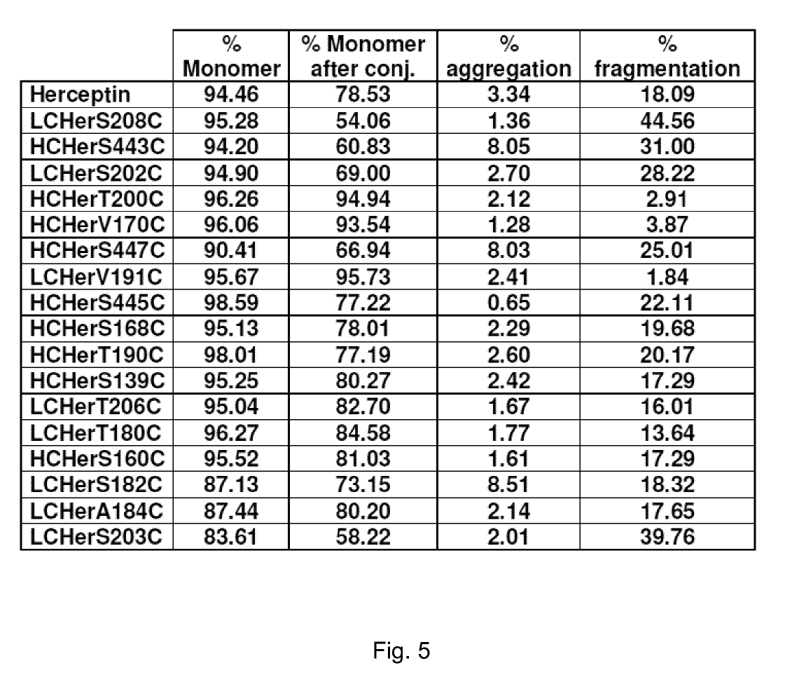

Larger scale cultures (250 ml) were prepared for further analysis. Results from SEC-HPLC analysis of conjugated and unconjugated samples are shown in FIGS. 5a and 5b, using samples at 1 mg/ml on a Zorbax-250 GF column. Standard analytical techniques (e.g. SEC HPLC, AUC, etc.) are used to assess aggregation propensity in vitro. The definition of “low” aggregation is based on SEC HPLC results, where the percentage monomer for the conjugated and non-conjugated molecule has to be over 70%, and/or ±10% of reference molecule.

The percentage of monomer lost after conjugation measured through size exclusion chromatography HPLC (high-performance liquid chromatography) is preferably ≤35%, more preferably ≤5%, ≤0%, ≤5%, ≤20%, ≤25%, or ≤30%. Percentage aggregation of the conjugated molecule is preferably <5%, <10%, <15%, or <20%, and the percentage fragmentation of the conjugated antibody is preferably <5%, <10%, <15%, <20%, <25%, <30%, <32%, <35% or <40%. Those conjugated variants with high aggregation and fragmentation are discarded (step S214).

### Binding Kinetics Assessment (In Vitro)

The introduction of a cysteine residue in the desired position should not interfere significantly with the binding of the molecule to its intended target, i.e. the binding kinetics should be comparable to a reference molecule (i.e. the parental antibody).

The conjugation of a payload to the engineered cysteine residue should not significantly impact the binding of the molecule to its intended target.

Binding kinetics of the variants were also analysed, using a quartz crystal microbalance. ERB2/HER2 Fc chimaera were immobilized to carboxyl chip, and three different concentrations of each variant (conjugated and not conjugated) were tested. The table below summarises the Kd for each variant:

K_{D} (nM)

| | Not Conjugated | Conjugated |
|---|---|---|
| Reference | | |
| Herceptin | 3.0 | 1.63 |
| Variant | | |
| LCHerS208C | — | — |
| HCHerS443C | 4.36 | 31.13 |
| LCHerS202C | 12.22 | 0.72 |
| HCHerT200C | 3.52 | 12.91 |
| HCHerV170C | 3.43 | 2.90 |
| HCHerS447C | 3.17 | 1.34 |
| LCHerV191C | 4.94 | 3.56 |
| HCHerS445C | 1.27 | 1.74 |
| HCHerS168C | 18.11 | 1.55 |
| HCHerT190C | 1.26 | 2.28 |
| HCHerS139C | 3.56 | 2.44 |
| LCHerT206C | 9.41 | 19.91 |
| LCHerT180C | 5.19 | 1.46 |
| HCHerS160C | 9.76 | 1.50 |
| LCHerS182C | 5.28 | 0.008 |
| LCHerA184C | 1.25 | 1.04 |
| LCHerS203C | — | — |

Preferably, the Constant of Dissociation (K_{D}) of the conjugated variants is 52 orders of magnitude than the reference standard K_{D} of an unmutated or parent antibody. For the antibodies described herein, the reference standard is herceptin (trastuzumab). Those conjugated variants with a K_{D} that falls outside this requirement are discarded from the selection process (step S216).

### Drug to Antibody Ratio (DAR) Assessment (In Vitro)

The DAR was determined for each of the variants, by LC-ESI-MS (liquid chromatography-electrospray ionisation-mass spectrometry). In embodiments, where there are two site specific conjugation sites per antibody, the DAR values for the different variants are preferably >1.7 and <2.2. More generally, for any number of site specific integration sites, the DAR values are preferably >85% and <110% of site specific conjugation sites per antibody. The variants which fall outside this range are discarded from the selection process (step S218).

Further filtering may be performed based on the non-specific binding properties of the antibody. Specific-binding is where the molecule binds to its intended target. Non-specific binding is where the molecule binds to targets that are not of interest, e.g anything other than the intended target. Preferably, the non-specific binding is minimal, to increase the possibility of the molecule binding to the intended target. In embodiments, the non-specific binding is ≤5%, ≤10%, ≤15%, ≤19%, ≤20%, ≤25%. More particularly, the non-specific binding on the heavy chain is preferably ≤5%, ≤10%, ≤15%, ≤19%, ≤20%, ≤25% and non-specific binding on the light chain is preferably ≤3%, or in embodiments, ≤5%, ≤10%, ≤15%, ≤19%, ≤20%, ≤25%.

When stability of the ADC is analysed in vitro, the percentage average loss of conjugated molecule over a period of 8 days should be <39%. For determining the DAR samples of an ADC in the example described herein, samples at 1 mg/ml were treated with PNGaseF. Reduced and not-reduced samples were analysed, by RP chromatography, electrospray, and mass spectrometry.

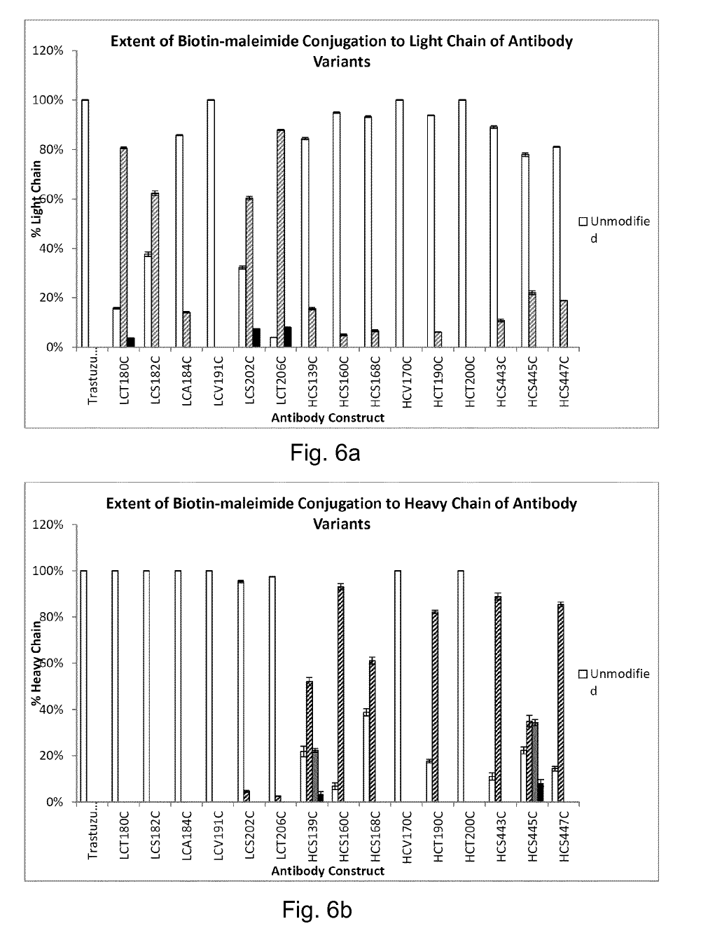

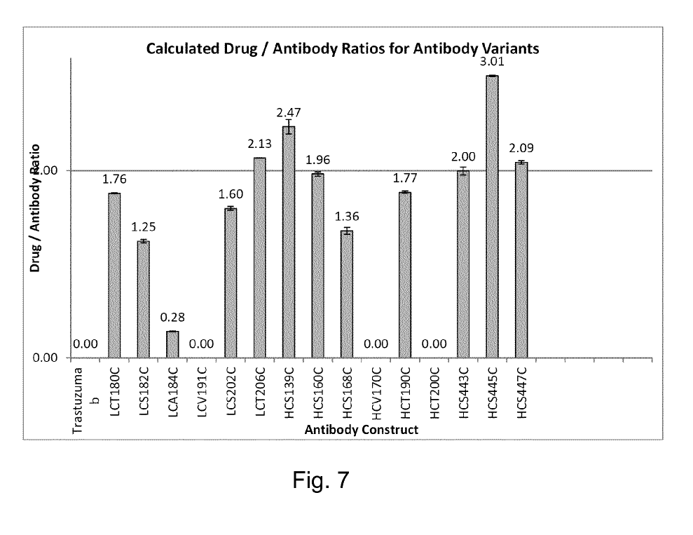

The extent of biotin-maleimide conjugation to light and heavy chains of the variants is shown in FIGS. 6a and 6b respectively, with the calculated DAR for each variant being shown in FIG. 7.

Six final antibody variants were selected as a result of following steps S200 to S218 of the selection process: S160C, T190C, S443C, S447C (on the heavy chain), T180C, or T2060 (on the light chain). As a result of this sequential method of selection all final variants share a number of specific properties (or design criteria): stability; low aggregation; low chemical degradation risk; low undesired post translational modifications; structural stability preserved; productivity; suitability for being conjugated; and biological activity. Some of these design criteria are described above, while the suitability of conjugation criteria are discussed below.

### Conjugation and De-Conjugation Assessment (In Vitro)

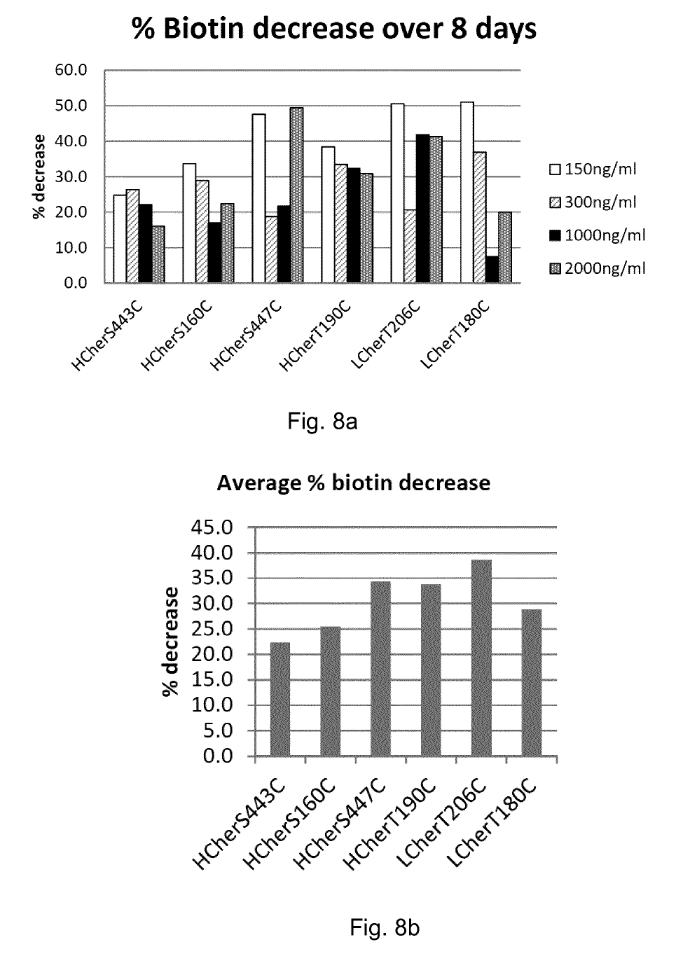

Each of these six variants was then further analysed. Conjugate stability (level of deconjugation) was determined for four different concentrations (150 ng/ml, 300 ng/ml, 1000 ng/ml and 2000 ng/ml) of each of the final variants in human serum at 37° C. for 8 days. Samples were taken on days 0, 2, 4 and 8, and analysed by ELISA. FIGS. 8a and 8b respectively show the percentage biotin decrease over 8 days and the average percentage biotin decrease for each variant.

Preferably, variant molecules have good reactivity—this might be parameterized with an upper and lower limit of the solvent accessibility surface (as per the in silico SAS criteria mentioned earlier), and validated in vitro.

The introduced cysteine residues in the variant antibodies are conjugated under standard reaction conditions (PBS pH 7.4).

Preferably, there is no di-sulphide scrambling. The introduction of a cysteine residue in the desired position should not lead to the formation of non-native disulfide bonds from disulfide scrambling

Preferably, conjugation occurs preferentially to the introduced cysteine residue in the desired position, with very little conjugation to native cysteine residues. Preferably, the specificity of conjugation to the introduced cysteine residue is >80% conjugation.

Preferably, conjugation reaction to the introduced cysteine residue is highly efficient, and minimises the amount of non-conjugated molecules, such that the conjugation efficiency is >80% (i.e. over 80% molecules conjugated under standard conditions, as above).

The modified molecule (antibody) preferably exhibits an average conjugate to antibody ratio as close as possible to 2. This implies a high specificity and high efficiency of conjugation. Preferably, the conjugate to antibody ratio is >1.7 and <2.2 where there are two site specific conjugation sites per antibody (or more generally, >85% and <110% of site specific conjugation sites).

It is generally important that there are low levels of de-conjugation in the conjugated variants, i.e. that the amount of payload that remains conjugated to the molecule after a period of time is relatively stable. In embodiments, after a period of 8 days in serum at 37° C., it is preferable that 60% of the conjugated product remains stable after 8 days.

Each of the final six variants was ranked for desirable characteristics (purity, DAR, deconjugation, and positive environment), and given a score from 6 (=best) to 1 (=worst). The scores were then totalled, to give an overall score from 4 to 24. This gives an indication of the desirability of each antibody for further development. The scores are shown in the table below:

| | | | | | % | | Positive | | |
|---|---|---|---|---|---|---|---|---|---|
| | Purity | | DAR | | Deconjugation | | Environment | | Total |
| HCHerS443C | 60.83 | 1 | 2.00 | 6 | 29 | 6 | LYS | 6 | 19 |
| HCHerS447C | 66.94 | 2 | 2.09 | 4 | 40 | 1 | | 2 | 9 |
| HCHerT190C | 77.19 | 3 | 1.77 | 2 | 37 | 3 | | 2 | 10 |
| LCHerT206C | 82.7 | 5 | 2.13 | 3 | 40 | 1 | LYS | 6 | 15 |
| LCHerT180C | 84.58 | 6 | 1.76 | 1 | 32 | 5 | | 2 | 14 |
| HCHerS160C | 81.03 | 4 | 1.96 | 5 | 32 | 5 | | 2 | 16 |

Although the antibody variants can be ranked in this way, as each has been through the initial selection process, they can all be said to have desirable characteristics for development as an ADC. In particular, not every antibody will make it through subsequent drug development processes and in vivo testing, so it is beneficial to be able to generate a selection of candidates. Nevertheless, while the six antibody variants above were selected from the 19 original variants following the above-described selection process and specific threshold values, the remaining 13 variants may also be useful under different conditions (e.g. if different threshold values are applied).

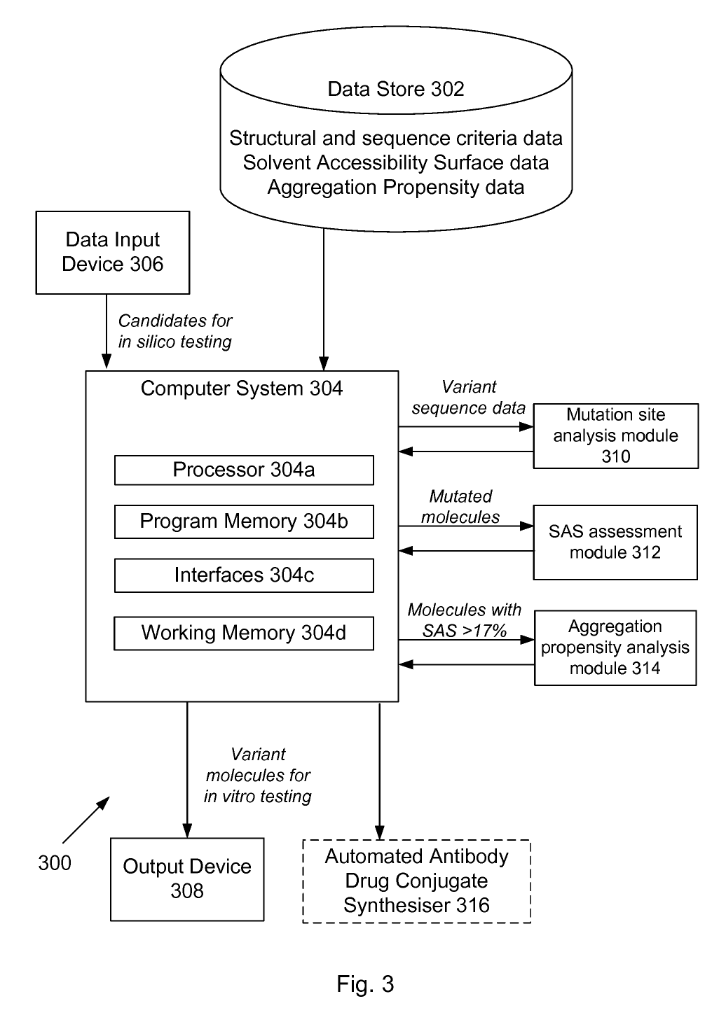

Referring now to FIG. 3, this shows a block diagram of a system 300 for implementing the above-described selection method, specifically for performing the in silico steps of the selection process. A general purpose computer system 304 comprises a processor 304a coupled to program memory 304b storing computer programme code to implement the method, to working memory 304d, and to interfaces 304c such as a conventional computer screen, keyboard, mouse, and printer, as well as other interfaces such as a network interface, and software interfaces such as a database interface.

The processor 304a may be an ARM™ device. The program memory 304b, in embodiments, stores processor control code to implement functions, including an operating system, various types of wireless and wired interface, storage, import and export from the device. The stored code also includes code to implement the method for selecting antibody variants as described above.

A user interface may also be provided, e.g. to enable a user to input candidate sequences into the computer system 304. A wireless interface, for example a Bluetooth™ or WiFi interface is optionally provided for interfacing with other devices, e.g. to receive the input candidate sequence. The wireless interface may comprise a Bluetooth™ RF chip and antenna.

The computer system 304 accepts user input from a data input device 306 such as a keyboard, input data file, or network interface, and provides an output to an output device 308 such as a printer, display, network interface, or data storage device. Input device 306, for example a network interface, receives an input comprising a candidate antibody sequence and provides this to the computer system 304 for in silico analysis. The output device 308 provides a list of variants of the candidate antibody sequence which have been selected following the above-described series of in silico tests.

Computer system 304 is coupled to a data store 302 which stores structural and sequence criteria data, solvent accessibility surface data and aggregation propensity data for each input candidate antibody sequence.

The computer system, in the illustrated example, is shown interfacing with a mutation site analysis module 310, a solvent accessibility surface (SAS) assessment module 312 and an aggregation propensity analysis module 314. One or more of these modules may be implemented as a separate machine, for example, coupled to computer system 304 over a network, or may comprise a separate or integrated programme running on computer system 304 (or on processor 304a). Whichever method is employed, in embodiments:
the mutation site analysis module 310 receives variant sequence data, applies the mutation site criteria described above (obtained from data store 302), and returns those variants which pass the mutation site criteria tests;
the SAS assessment module 312 receives the variant sequences which have passed the mutation site analysis, applies the SAS criteria (obtained from data store 302) and returns those variants which have SAS >17%, as described above; and
the aggregation propensity analysis module 314 receives the variants with SAS >17%, applies the aggregation propensity criteria (obtained from data store 302) and returns those variants which have a Z-score lying outside (−1, 1), as described above.

As illustrated, computer system 304 may also provide data to an output device 308. In this way computer system 304 may be programmed to automatically compare the properties of a number of antibody candidates, and select one or more of those which are predicted to have favourable properties for conjugation with a target payload. Additionally or alternatively, computer system 304 may provide a data output to an automated antibody drug conjugate synthesiser 316. In this way, the computer system may be programmed to automatically select mutated antibody drug sequences which are predicted to conjugate to a particular payload, and to output these sequences to the synthesiser 316 for expression and conjugation to the payload. An example of a suitable automated synthesiser is the ABI 433A Peptide Synthesiser. In embodiments, the output is simply the protein sequence which is taken into expression, through purification and then conjugation. Additionally or alternatively, the output of these sequences could be a DNA molecule encoding the protein sequence. This output could be automated, such that the protein sequences could be automatically sent from the computer system 304 to a synthesiser for DNA optimisation and synthesis.

No doubt many other effective alternatives will occur to the skilled person. It will be understood that the invention is not limited to the described embodiments and encompasses modifications apparent to those skilled in the art lying within the spirit and scope of the claims appended hereto.

1. A computer-implemented method of identifying one or more antibody sequences which are predicted to permit conjugation to a payload, each antibody sequence having a variable region which binds a target molecule and a constant region which comprises a mutated residue introducing a site specific conjugation site to permit conjugation of the antibody to a payload, the method comprising:
inputting a candidate antibody sequence;
identifying, using a mutation site analysis module, a plurality of mutated antibody sequences by:
identifying at least one site in the candidate antibody sequence where a mutated residue is introducible;
identifying at least one mutation which is introducible at each identified site; and
introducing each identified mutation at each identified site to produce a plurality of mutated antibody sequences;
calculating, using a solvent accessibility surface module, a value representative of solvent accessibility surface for each of the plurality of mutated antibody sequences;
calculating, using an aggregation propensity module, a value representative of aggregation propensity for each of the plurality of mutated antibody sequences;
comparing the calculated solvent accessibility value and the aggregation propensity value with the corresponding threshold values for solvent accessibility surface and aggregation propensity;
filtering the plurality of mutated antibody sequences based on whether the threshold values for solvent accessibility and aggregation propensity are met; and
outputting the filtered mutated antibody sequences which are predicted to permit conjugation to a payload.

2. The method of claim 1 wherein the value representative of solvent accessibility surface for a specific mutated amino acid residue is a predicted percentage side chain solvent accessibility surface ratio which is given by:
$$100 \times \frac{S}{R}$$
where S is side chain solvent accessibility for a candidate amino acid residue, and R is side chain solvent accessibility of the mutated amino acid residue.

3. The method of claim 1 wherein the threshold value for solvent accessibility surface is >15%.

4. The method of claim 1 wherein the outputting step comprises outputting mutated sequences having a predicted percentage side chain solvent accessibility surface of greater than the threshold value.

5. The method of claim 1 wherein the threshold value for aggregation propensity is calculated using a mean aggregation propensity for a set of reference antibody sequences, and wherein the outputting step comprises outputting mutated sequences which are predicted to have an aggregation propensity less than or equal to the threshold value, wherein preferably the method further comprises calculating a standard deviation value for the set of reference antibody molecules, wherein the outputting step comprises outputting mutated antibody sequences which are predicted to have an aggregation propensity value within one standard deviation from the mean aggregation propensity.

6. (canceled)

7. The method of claim 1 further comprising identifying immunogenicity propensity of the mutated antibody sequences, wherein preferably identifying immunogenicity propensity comprises:
predicting immunogenicity propensity of each mutated antibody; and
comparing the predicted immunogenicity propensity of each mutated antibody with immunogenicity propensity of the candidate antibody;
wherein the outputting step comprises outputting mutated antibody sequences having a substantially similar immunogenicity propensity to the candidate antibody.

8. (canceled)

9. The method of claim 1 wherein identifying the plurality of mutated antibody sequences further comprises:
predicting physiochemical properties of each mutated antibody sequence;
comparing the predicted physiochemical properties to physiochemical properties of the candidate antibody sequence; and
selecting mutated antibody sequences having predicted physiochemical properties which are substantially similar to the physiochemical properties of the candidate antibody.

10. The method of claim 1 wherein filtering the mutated antibody sequences based on the solvent accessibility surface value is performed before filtering the mutated antibody sequences based on the aggregation propensity value and wherein preferably, filtering the mutated antibody sequences based on aggregation propensity value is performed before filtering the mutated antibody sequences based on solvent accessibility surface value.

11. (canceled)

12. A method of making an antibody for drug conjugation comprising:
selecting one of the mutated antibody sequences which is output from the method of claim 1;
synthesising the sequence; and
conjugating a payload to the selected, synthesised sequence.

13. A method of selecting one or more antibody drug conjugates, comprising:
making at least one antibody drug conjugate using the method of claim 12;
performing one or more in vitro tests to determine biological and physiochemical properties of the antibody drug conjugates;
comparing the determined biological and physiochemical properties with corresponding threshold values for the biological and physiochemical properties; and
selecting the antibody drug conjugates based on whether the threshold values for the biological and physiochemical properties are met.

14. A method of selecting as claimed in claim 13 wherein the physiochemical properties include a drug to antibody ratio (DAR) having a threshold value of between 85% and 110% of site specific conjugation sites per antibody, and wherein the selecting step comprises selecting antibody drug conjugates having a determined DAR value within the threshold value range, wherein preferably the antibody drug conjugates comprise two introduced site specific conjugation sites and the threshold value for DAR is between 1.7 to 2.2, and wherein the selecting step comprises selecting antibody drug conjugates having a determined DAR value within the threshold value range.

15. (canceled)

16. The method of claim 13 wherein the physiochemical properties include a value for conjugation efficiency, wherein the threshold value for conjugation efficiency is at least 80%, and wherein the selecting step comprises selecting antibody drug conjugates having a determined conjugation efficiency greater than or equal to the threshold value.

17. The method of claim 13 wherein the physiochemical properties include a value for stability, wherein the threshold value for stability is at least 60% for a pre-defined period of time, and wherein the selecting step comprises selecting antibody drug conjugates having a determined stability greater than or equal to the threshold value.

18. A system for identifying one or more antibody sequences which are predicted to permit conjugation to a payload, each antibody sequence having a variable region which binds a target molecule and a constant region which comprises a mutated residue introducing a site specific conjugation site to permit conjugation of the antibody to a payload, the system comprising:
an input device for inputting a candidate antibody sequence;
a database comprising solvent accessibility surface and aggregation propensity data and corresponding threshold values thereof;
at least one processor coupled to the input device and the database, wherein the processor is configured to:
receive the input candidate antibody sequence;
identify, using a mutation site analysis module, a plurality of mutated antibody sequences by:
identifying at least one site in the candidate antibody sequence where a mutated residue is introducible;
identifying at least one mutation which is introducible at each identified site; and
introducing each identified mutation at each identified site to produce a plurality of mutated antibody sequences;
calculate, using a solvent accessibility surface module, a value representative of solvent accessibility surface for each of the plurality of mutated antibody sequences;
calculate, using an aggregation propensity module, a value representative of aggregation propensity for each of the plurality of mutated antibody sequences;
compare the calculated solvent accessibility surface value and the aggregation propensity value with the corresponding threshold values for solvent accessibility surface and aggregation propensity;
filter the plurality of mutated antibody sequences based on whether the threshold values for solvent accessibility and aggregation propensity are met; and
output the filtered mutated antibody sequences which are predicted to permit conjugation to a payload.

19. The system of claim 18 wherein the solvent accessibility surface module is configured to:
receive the identified plurality of mutated antibody sequences from the processor;
request the solvent accessibility surface threshold value from the database; and
calculate the value representative of solvent accessibility surface for each mutated antibody sequence;
wherein the value representative of solvent accessibility surface is a predicted percentage side chain solvent accessibility surface ratio for each mutated antibody sequence given by:
$$100 \times \frac{S}{R}$$
where S is side chain solvent accessibility for a candidate amino acid residue, and R is side chain solvent accessibility of a specific mutated antibody sequence.

20. The system of claim 18 wherein the threshold value for solvent accessibility surface is 17%, and wherein the solvent accessibility surface module is configured to:
filter the received identified plurality of mutated antibody sequences; and
output those mutated antibody sequences having a solvent accessibility surface value greater than the threshold value.

21. The system of claim 18 wherein the aggregation propensity analysis module is configured to:
receive the identified plurality of mutated antibody sequences from the processor;
request a set of reference antibody sequences from the database;
calculate the aggregation propensity threshold value by calculating the mean aggregation propensity for the set of reference antibody sequences;
calculate a value representative of aggregation propensity for each received mutated antibody sequence;
filter the received identified plurality of mutated antibody sequences; and
output those mutated antibody sequences having an aggregation propensity value less than or equal to the threshold value.

22. The system of claim 21 wherein the aggregation propensity analysis module is further configured to:
calculate a standard deviation value for the set of reference antibody sequences; and
filter the received identified plurality of mutated antibody sequences; and
output those mutated antibody sequences having an aggregation propensity value within one standard deviation from the mean aggregation propensity.

23. The system of claim 18 wherein the at least one processor is configured to filter the mutated antibodies based on solvent accessibility surface criteria before filtering the mutated antibodies based on aggregation propensity criteria.

24. The system of claim 18 wherein the at least one processor is configured to filter the mutated antibodies based on aggregation propensity criteria before filtering the mutated antibodies based on solvent accessibility surface criteria.

25. A method of making a pharmaceutical composition wherein the composition comprises at least one antibody or antibody fragment having a variable region which binds a target molecule and a constant region, wherein the constant region comprises one or more mutations introducing a site specific conjugation site to permit conjugation of the antibody to a payload, wherein the at least one antibody or antibody fragment is identified by the method of claim 1, and the composition is formulated with a pharmaceutically acceptable carrier, adjuvant and/or excipient.

26. A carrier carrying code which when implemented on a processor causes said processor to carry out the steps of claim 1.

27. A method of identifying one or more antibodies substantially as hereinbefore described with reference to FIGS. 2a and 2b.

28. A system of identifying one or more antibodies substantially as hereinbefore described with reference to FIG. 3.
